(19) United States
(12) Patent Application Publication
(10) Pub. No.: US 2003/0181772 A1
(43) Pub. Date: Sep. 25, 2003

(54) Method and device for treating a c4 fraction

(76) Inventors: Gerald Meyer, Ludwigshafen (DE); Gerd Kaibel, Lampertheim (DE); Gerd Bohner, Malsch (DE); Klaus Kindler, Harthausen (DE); Till Adrian, Bobenheim-Roxheim (DE); Karin Pickenaecker, Lampertheim (DE); Melanie Pahl, Mannheim (DE); Thomas Hill, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,898

(22) PCT Filed: May 9, 2001
(86) PCT No.: PCT/EP01/05279

(30) Foreign Application Priority Data
May 9, 2000 (DE) 100 22 465.2

(51) Int. Cl. C07C 5/00; C07C 5/02

(52) U.S. Cl. 585/324; 585/258; 585/259; 585/833

(57) ABSTRACT

In a process for the work-up of a C4 fraction, comprising the process steps

Extractive distillation (I),

Selective hydrogenation over a heterogeneous catalyst (II), a crude 1,3-butadiene stream being obtained following process steps (I) and (II) and

Distillation of the crude 1,3-butadiene stream for isolating pure 1,3-butadiene (III),

the process steps I and II are carried out in a single column or in thermally coupled columns and the process step III is carried out in a second column.

The present invention relates to a process for isolating 1,3-butadiene by work-up of a C4 fraction and to an apparatus for carrying out the process. The C4 fraction obtained in crackers, is a mixture of hydrocarbons in which C4-hydrocarbons, in particular 1-butene, i-butene and 1,3-butadiene, predominate. Apart from small amounts of C3- and C5-hydrocarbons, the C4 fraction generally further comprises C3- and C4-acetylenes, for example 1-butyne, butenyne and propyne, in particular 1-butyne (ethylacetylene) and butenyne (vinylacetylene).

The isolation of 1,3-butadiene from such mixtures represents a complicated distillation problem because of the small differences in the relative volatilities. Fractionation is therefore carried out by extractive distillation, i.e. distillation with addition of an extractant which has a boiling point higher than that of the mixture to be fractionated and increases the differences in the relative volatilities of the components to be separated. When suitable extractants are used, the abovementioned C4 fraction can be fractionated by extractive distillation to give a crude 1,3-butadiene fraction which is subsequently purified further in final distillation columns together with a stream comprising hydrocarbons having a lower solubility than 1,3-butadiene, in particular butanes and butenes, and a stream comprising hydrocarbons which are more readily soluble than 1,3-butadiene, in particular butynes and possibly 1,2-butadiene. Such a process is described, for example, in EP-B 0 284 971. However, it has the disadvantage that the components which are more soluble in the extractant than is 1,3-butadiene, in particular the butenynes and possibly 1,2-butadiene, are not converted into the desired product 1,3-butadiene.

A further disadvantage is a loss of raffinate, since the acetylene-rich stream has to be diluted with raffinate 1 for safety reasons.

The extractive distillation for isolating 1,3-butadiene can be simplified by prior selective hydrogenation of acetylenic impurities, i.e. the butynes. Such a process is described in Proc.-Ethylene Prod. Conf. 5 (1996), pages 631 to 636. According to this, a high vinylacetylene conversion combined with a low butadiene loss is achieved at high catalyst operating lives when using a KLP catalyst, i.e. a catalyst which comprises finely divided copper particles on a high-purity γ-aluminum oxide having a defined pore structure as support. The prior selective hydrogenation enables the two-stage extractive butadiene distillation to be simplified to a single-stage process and enables the equipment items required in the downstream final distillation to be reduced by one separation column. However, the process has the disadvantage that a separate plant is necessary for the prior selective hydrogenation of the acetylenic impurities.

U.S. Pat. No. 4,277,313 discloses a further process for isolating 1,3-butadiene, according to which firstly a selective hydrogenation and then an extractive distillation of the 1,3-butadiene are carried out. The selective hydrogenation can be carried out in the liquid phase or in the gas phase, in the presence of catalysts comprising elements of group VIII of the Periodic Table, for example a palladium/aluminum oxide catalyst. Extractants mentioned are dimethylformamide or diethylformamide, N-methylpyrrolidone, furfural or acetonitrile. The process has, like the process described above, the disadvantage that a separate plant is necessary for the prior selective hydrogenation.

U.S. Pat. No. 6,040,489 discloses a process for separating 1,3-butadiene from a C4 fraction in which the C4 fraction is hydrogenated in a column and selectively extracted with a solvent, a stream comprising at least butanes and butenes is taken off from the column as a top stream and the solvent, laden with butadienes, is taken off at the bottom and then separated in a solvent stripping column into a butadiene-containing top stream and a solvent-containing bottom stream. In a butadiene distillation column, the butadiene-containing top stream is separated into a 1,3-butadiene-containing top stream and a 1,2-butadiene-containing bottom stream.

The use of dividing wall columns, i.e. distillation columns having vertical dividing walls which in regions of the column prevent crossmixing of liquid and vapor streams, for the fractionation of multicomponent mixtures by distillation is known. The dividing wall, which comprises a flat metal sheet, divides the column in the longitudinal direction in its middle region into an inflow section and an offtake section.

A similar result can be achieved using thermally coupled columns, i.e. arrangements of at least two columns in which each of the columns is connected to each other column and at least two physically separate connection points.

EP-B 0 126 288 describes a dividing wall column in which chemical reactions are carried out. As a result of defined addition of homogeneous catalysts, chemical reactions can be restricted in a targeted manner to particular regions of the dividing wall column.

It is an object of the present invention to provide a process for isolating 1,3-butadiene from a C4 fraction, which process does not have the disadvantages of the prior art, in particular requires a lower outlay in terms of apparatus.

In the present instance the term crude 1,3-butadiene refers to a hydrocarbon mixture containing the target product 1,3-butadiene in a fraction of at least 80% by weight, preferably 90% by weight, with particular preference 95% by weight, the remainder made up of impurities.

In contrast, the term pure 1,3-butadiene refers to a hydrocarbon mixture containing the target product 1,3-butadiene in a fraction of at least 99% by weight, preferably 99.5% by weight, with particular preference 99.7% by weight, the remainder made up of impurities.

The achievement of this object starts out from a process for the work-up of a C4 fraction, comprising the process steps

Extractive distillation (I),

Selective hydrogenation over a heterogeneous catalyst (II), a crude 1,3-butadiene stream being obtained following process steps (I) and (II) and

Distillation of the crude 1,3-butadiene stream for isolating pure 1,3-butadiene (III).

We have found that the abovementioned object is achieved by the process steps I and II being carried out in a single column and the process step III being carried out in a second column.

As an alternative, it is possible to carry out the process steps I and II in thermally coupled columns and to carry out the process step III in a second column.

The known processes give no indications that a C4 cut could be carried out by extractive distillation and selective hydrogenation in heterogeneous catalysis to give a crude 1,3-butadiene stream in a single column. The assumption, on the contrary, was that an additional apparatus, a stripping column in particular, was necessary for separating 1,3-butadiene from the selective solvent laden with it, and that such an apparatus, a stripping column in particular, would have to be operated under different process conditions, in particular under different pressure conditions. The reason for this lies in the strong polymerization propensity of the dienic and acetylenic compounds at elevated temperature. These increases in temperature occur in the lower region of the column and in the evaporator if the low-boiling hydrocarbons are separated by distillation from the high-boiling extractant under the pressure conditions of the extractive distillation, i.e. at from about 4 to 6 bar absolute.

The C4 fraction, as it is known, which is to be used in the present case as the starting mixture is a mixture of hydrocarbons having predominantly four carbon atoms per molecule. C4 fractions are obtained, for example, in the production of ethylene and/or propylene by thermal cracking of a petroleum fraction such as liquefied petroleum gas, light naphtha or gas oil. C4 fractions are also obtained in the catalytic dehydrogenation of n-butane and/or n-butene. C4 fractions generally include butanes, butenes, 1,3-butadiene, and also small amounts of C3- and C5-hydrocarbons, and also butynes, especially 1-butyne (ethyl acetylene) and butenyne (vinyl acetylene). The 1,3-butadiene content is generally from 10 to 80% by weight, preferably from 20 to 70% by weight, in particular from 30 to 60% by weight, while the amount of vinyl acetylene and ethyl acetylene generally does not exceed 5% by weight.

A typical C4 fraction has the following composition in percent by weight:

| | |
|---|---|
| Propane | 0-0.5 |
| Propene | 0-0.5 |
| Propadiene | 0-0.5 |
| Propyne | 0-0.5 |
| n-Butane | 3-10 |
| i-Butane | 1-3 |
| 1 -Butene | 10-20 |
| i-Butene | 10-30 |
| trans-2-Butene | 2-8 |
| cis-2-Butene | 2-6 |
| 1,3-Butadiene | 30-60 |
| 1,2-Butadiene | 0.1-1 |
| Ethylacetylene | 0.1-2 |
| Vinylacetylene | 0.1-3 |
| C5 | 0-0.5 |

In the extractive distillation just defined, suitable selective solvents for the present separation problem, namely the isolation of 1,3-butadiene from the C4 fraction, are generally substances or mixtures which have a boiling point higher than that of the mixture to be fractionated and have a greater affinity for conjugated double bonds and triple bonds than for simple double bonds and single bonds, preferably dipolar solvents, particularly preferably dipolar aprotic solvents. Substances which are not corrosive or only slightly corrosive are preferred so as to avoid attack on the apparatus.

Suitable selective solvents for the process of the present invention are, for example, butyrolactone, nitriles such as acetonitrile, propionitrile or methoxypropionitrile, ketones such as acetone, furfural, N-alkyl-substituted lower aliphatic acid amides such as dimethylformamide, diethylformamide, dimethylacetamide, diethylacetamide or N-formylmorpholine, N-alkyl-substituted cyclic acid amides (lactams) such as N-alkylpyrrolidones, in particular N-methylpyrrolidone. Use is generally made of N-alkyl-substituted lower aliphatic acid amides or N-alkyl-substituted cyclic acid amides. Particularly advantageous extractants are dimethylformamide and, in particular, N-methylpyrrolidone.

However, it is also possible to use mixtures of these solvents with one another, for example N-methylpyrrolidone with acetonitrile, or mixtures of these solvents with cosolvents such as water and/or tert-butyl ethers, for example methyl tert-butyl ether, ethyl tert-butyl ether, propyl tert-butyl ether, n- or isobutyl tert-butyl ether.

A particularly useful extractant is N-methylpyrrolidone, preferably in aqueous solution, in particular with from 8 to 10% by weight of water, particularly preferably with 8.3% by weight of water.

For the selective hydrogenation over heterogeneous catalysts, namely process step II, essentially all known processes can be used for the purposes of the present invention. It is possible to use the known catalysts based on palladium as are described, for example, in EP-A-0 738 540, EP-A-0 722 776 or U.S. Pat. No. 4,587,369, or catalysts based on copper as described, for example, in U.S. Pat. No. 4,493,906 or U.S. Pat. No. 4,704,492.

The catalysts for the selective hydrogenation can be applied to customary distillation internals, i.e., in particular, column trays, shaped bodies or packings; they can be embedded in pockets of wire mesh and wound up into rolls, as described in U.S. Pat. No. 4,215,011. However, they are particularly advantageously used as TLC (Thin Layer Catalyst) packings.

Particularly useful forms of catalysts are the TLC catalyst packings described in DE-A 196 24 130 and obtained by vapor deposition and/or sputtering; the contents of this publication are hereby fully incorporated by reference into the disclosure of the present invention. In addition to the woven meshes or films described in DE-A 196 24 130 as support material, it is also possible to use a knitted mesh as support material for the catalyst packing. In addition to the vapor deposition and/or sputtering described in DE-A 196 24 130, the catalytically active substances and/or substances active as promoter can also be applied by impregnation.

The distillation of the crude 1,3-butadiene stream for the purpose of recovering pure 1,3-butadiene (process step III) takes place in a second distillation column, in a known way, in particular in a dividing wall column or in one column or in 2 columns. The feed stream for process step III is preferably withdrawn from the first column in the form of a vaporous sidestream and supplied to the second distillation column.

As far as the columns which can be used to implement process steps I and II to give a crude 1,3-butadiene stream are concerned, there are in principle no restrictions.

The column is supplied in its middle region with the C4 fraction, the selective solvent in its upper region, and hydrogen below the C4 fraction supply side.

The column is equipped with separation-active internals, which are preferably random packing elements or ordered packings in the region below the selective solvent supply side. Above the selective solvent supply side it is preferred to arrange one or more trays.

The column is preferably operated at a column-top pressure in the range from 3 to 7 bar absolute, in particular from 4 to 6 bar absolute; by this means it is possible to carry out condensation with water as coolant at the top of the column, without any need for more expensive coolants.

In the column bottom, temperatures in the range from about 140 to 200° C., in particular from 180 to 190° C., frequently of about 185° C., become established.

At least the separation-active internals below the C4 fraction supply side, particularly random packing elements or ordered packings, are configured as reactive internals, in other words catalysts for the selective hydrogenation are applied to them, as already described above. Preference is given to using TLC packings.

Taken off at the top of the column is a stream which comprises those components of the C4 fraction which are less soluble than 1,3-butadiene in the selective solvent, particularly butanes and butenes, while from the column bottom selective solvent is taken off, still contaminated with hydrocarbons, which are preferably separated off in a vaporizer and supplied to the column bottom again to give a purified solvent which preferably is at least partly recycled into the upper region of the column.

In one preferred process variant a stream is taken off from the column in which process steps (I) and (II) are conducted from a zone having a relatively high concentration of acetylenes and this stream is supplied again to the column, preferably in the topmost region of the catalytically active zone of said column. This produces an increase in the yield of 1,3-butadiene.

It is preferred to conduct the process in such a way that at least one measure is taken to lower the temperature of the liquid in the bottom of the column. In accordance with this process variant, the temperature to which the reaction mixture is subjected is reduced.

The temperature of the liquid in the bottom of the column is reduced preferably by from 10 to 80° C., in particular to a level in the range from 100 to 170° C., preferably from 140 to 160° C.

One preferred measure for lowering the temperature of the liquid in the bottom of the column is, in accordance with the invention, to supply a stream of middle boilers to the lower column region or to the bottom vaporizer of the column. The term “middle boilers” refers in the present case to a hydrocarbon or a mixture of hydrocarbons which is defined by way of its boiling point:

Said boiling must in the present case be situated above the boiling point of 1,3-butadiene and below the boiling point of the solvent or solvent mixture.

The middle boilers supplied preferably comprises a substance or a mixture of substances which is already present in the process.

Particularly suitable middle boilers comprise a substance or a mixture of substances having in each case 5 carbon atoms per molecule, preferably one or more alkanes and/or one or more alkenes.

As middle boilers it is particularly preferred to supply one or more of the substances 2-methyl-2-butene, 3-methyl-1-butene, n-pentane, isopentane, n-pent-1-ene and n-pent-2-ene.

The ratio of the volume flow of the middle boiler to the volume flow of the C4 fraction supplied is preferably from 0.001/1 to 0.25/1, more preferably from 0.002/1 to 0.15/1, with particular preference from 0.004/1 to 0.008/1.

As the middle boiler stream it is also possible in particular to supply a bottom stream from the distillation column for recovering pure 1,3-butadiene.

A second measure which can be taken in accordance with the invention in order to lower the temperature of the liquid in the bottom of the column, in addition to or alternatively to the above-described supply of a middle boiler stream, is to raise the amount of relatively low-boiling components from the selective solvent, in particular its water vapor content, in the lower region of the column by supplying a stream of the relatively low-boiling component of the selective solvent, steam in particular, to the lower region of the column and depleting the stream of selective solvent taken off from the column, prior to its partial or complete recycling to the column, by the supplied fraction of relatively low-boiling component, in particular steam.

The ratio of the relative volume flow of relatively low-boiling component, especially steam, to the volume flow of the C4 fraction supplied to the column is preferably from 0.2/1 to 1.6/1, preferably 1.2:1.

The relatively low-boiling component of the selective solvent, especially water, is appropriately supplied to the column in vapor form, preferably at a pressure equal to or slightly above the bottom pressure of the column.

A selective solvent which is particularly suitable in the present process is, as set out above, N-methylpyrrolidone, referred to for short as NMP, preferably in aqueous solution, in particular with from 8 to 10% by weight of water, with particular preference with 8.3% by weight of water.

Prior to the recycling of the selective solvent stream it can be depleted by the supplied fraction of relatively low-boiling component, especially steam. This entails little or no change to the composition of the selective solvent, which is essential for its selectivity.

As an alternative to the measures described above, the temperature in the bottom of the column can be lowered by allowing an increased 1,3-butadiene content in the bottoms liquid of the column, in particular from 0.5 to 5% by weight based on the total weight of the bottoms liquid, preferably from 1 to 3% by weight, with particular preference 1.8% by weight, and depleting the bottoms liquid, after it has been taken off from the column, of 1,3-butadiene in a stripping column, using as stripping vapor preferably the vaporous top product of the column.

It is also possible to equip the stripping column with an additional bottoms vaporizer.

Alternatively it is possible to carry out the butadiene depletion not in a separate stripping column but instead in an additional subsection arranged in the lowermost region of the column.

In one embodiment, the process steps I and II are carried out in a dividing wall column.

For this purpose, use is made of an apparatus comprising

a dividing wall column in which a dividing wall is arranged in the longitudinal direction of the column to form an upper common column region, a lower common column region, an inflow section and an offtake section,

with introduction of the feed mixture in the middle region of the inflow section, introduction of the extractant in the upper region of the inflow section,

with introduction of hydrogen in the lower common column region, separation of unreacted hydrogen in the vapor stream from condensable low boilers in a condenser at the top of the dividing wall column and recirculation via a compressor to the lower common column region and

with liquid or vapor discharge of the crude 1,3-butadiene stream from the offtake section of the dividing wall column at a point below the corresponding feed point in the inflow section and

further transport of the crude 1,3-butadiene stream to the final distillation (process step III).

The starting mixture, namely the C4 fraction, is vaporized beforehand and fed in vapor form into the middle region of the inflow section of the dividing wall column. The extractant is introduced in the upper region of the inflow section of the dividing wall column at a feed point selected so that it is sufficiently far below the upper end of the dividing wall to ensure that no extractant gets into the upper common column region wall and into the upper region of the offtake section.

In the condenser located at the top of the dividing wall column, the condensable low boilers, in particular butanes, butenes and possibly C3-hydrocarbons are condensed from the vapor stream and are preferably partly returned as runback to the top of the dividing wall column and otherwise discharged as low boiler stream. The hydrogen which has not been consumed in the hydrogenation is compressed in a compressor and fed in gas form back to the lower common column section. Hydrogen which has been consumed is replaced by fresh hydrogen. However, as an alternative to or in addition to the recirculation of the hydrogen which has not been consumed into the lower common column region, the unused hydrogen can be recirculated via the bottom vaporizer of the column. Feeding the hydrogen into the bottoms vaporizer offers the advantage of a significant lowering of the temperature of the bottom product and allows better separation of the hydrocarbons from the bottom product without the maximum permissible operating temperature for the extractant being exceeded.

The crude 1,3-butadiene stream is taken off in vapor or liquid form from the lower region of the offtake section of the dividing wall column at a point which is located below the corresponding feed point for the C4 fraction in the inflow section. Here, the discharge point has to be sufficiently far above the lower end of the dividing wall to ensure that no extractant can get from the lower common column region into the region of the offtake section above the discharge point for the 1,3-butadiene-containing stream.

All regions of the column can be provided with customary distillation internals. In addition, at least one region of the offtake section has to be provided with reactive internals, i.e. with internals which heterogeneously catalyze the selective hydrogenation. For this purpose, it is possible, as indicated above, to use customary distillation internals to which the heterogeneous catalysts have been applied or preferably TLC packings. In addition to the above-defined subsection of the offtake section, the entire upper subsection of the offtake section can also be provided with reactive internals.

In a further embodiment, the present invention provides an apparatus for carrying out the process of the present invention in which the dividing wall column is replaced by thermally coupled columns, preferably each having their own bottoms vaporizer and/or condenser.

These assemblies are equivalent in terms of energy consumption to a dividing wall column. These apparatus variants make it possible to operate the two columns at different pressures. Since the hydrogen partial pressure for the selective hydrogenation is from about 1 to 10 bar, the parts of the plant in which hydrogen is present have to be designed for a correspondingly elevated pressure. The use of thermally coupled columns of which only one has to be designed for the elevated operating pressure enables the capital costs to be reduced. The apparatus variants using thermally coupled columns also offer advantages when catalysts having a short operating life are used. Location of the catalyst in a side column makes it possible to provide two such columns connected in parallel so that downtimes for catalyst regeneration, catalyst washing or catalyst replacement can either be completely avoided or at least substantially reduced.

Dividing wall columns are preferred in new plants for cost reasons, but thermally coupled columns are useful, in particular, for the modification of existing distillation columns.

The invention is illustrated below with the aid of a drawing and examples. In the drawing:

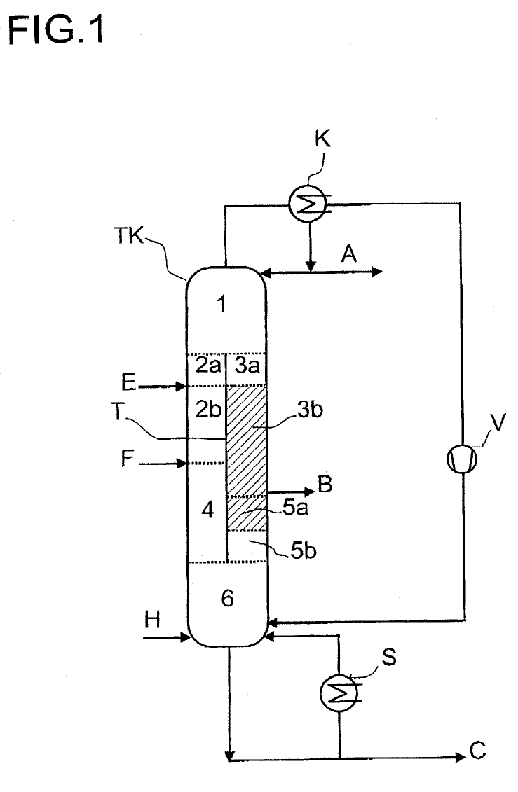

FIG. 1 schematically shows a first apparatus according to the present invention comprising a dividing wall column,

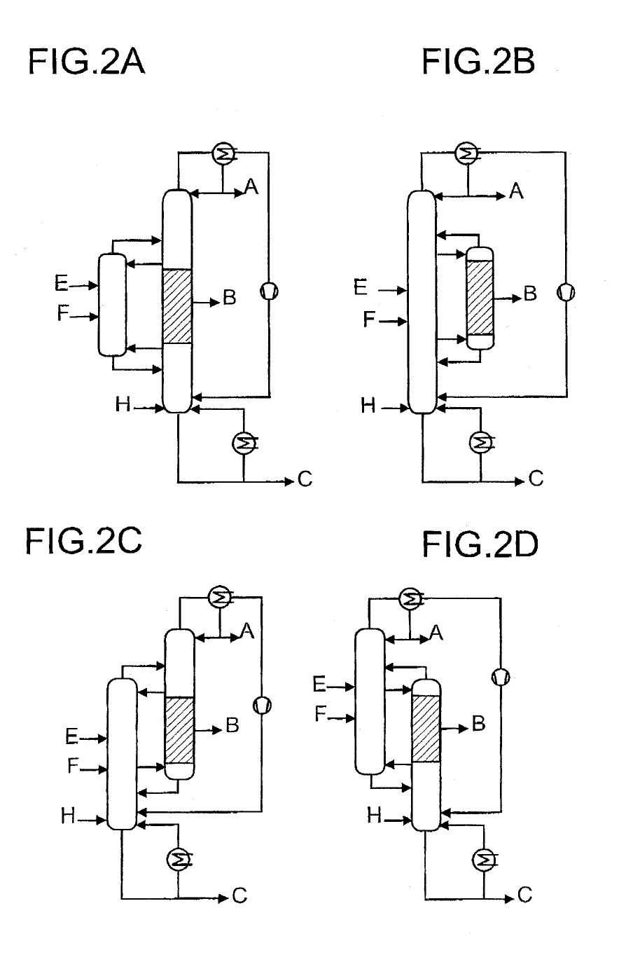

FIGS. 2a to 2d schematically show thermally coupled columns with common bottoms vaporizer and condenser and

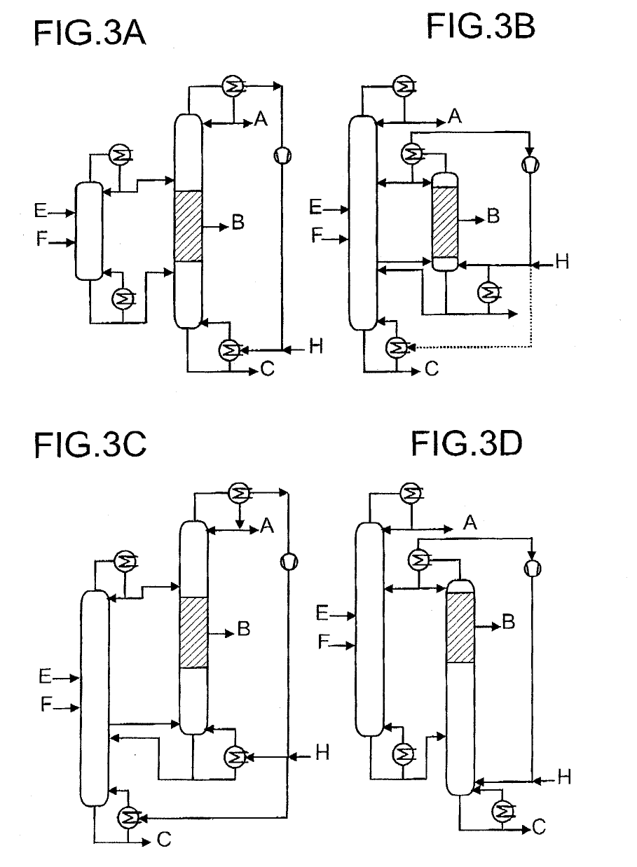

FIGS. 3a to 3d schematically show thermally coupled columns each having their own bottoms vaporizer and condenser.

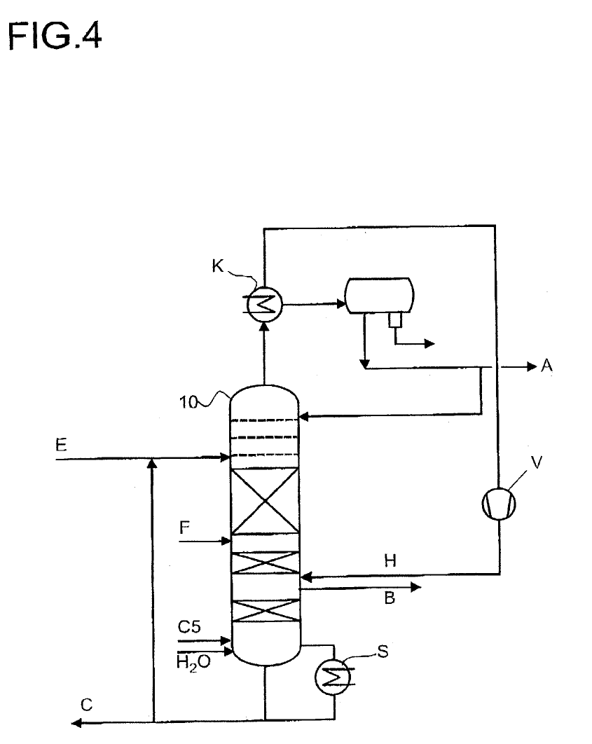

FIG. 4 schematically shows a process variant with supplying of a middle boiler stream,

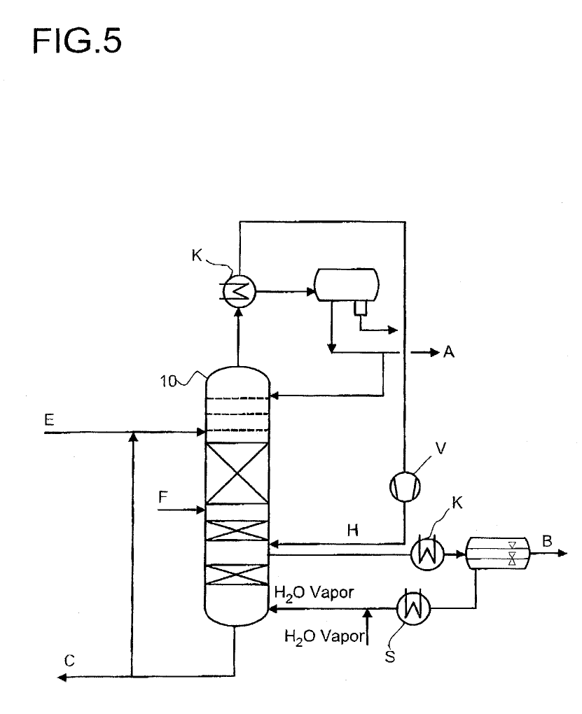

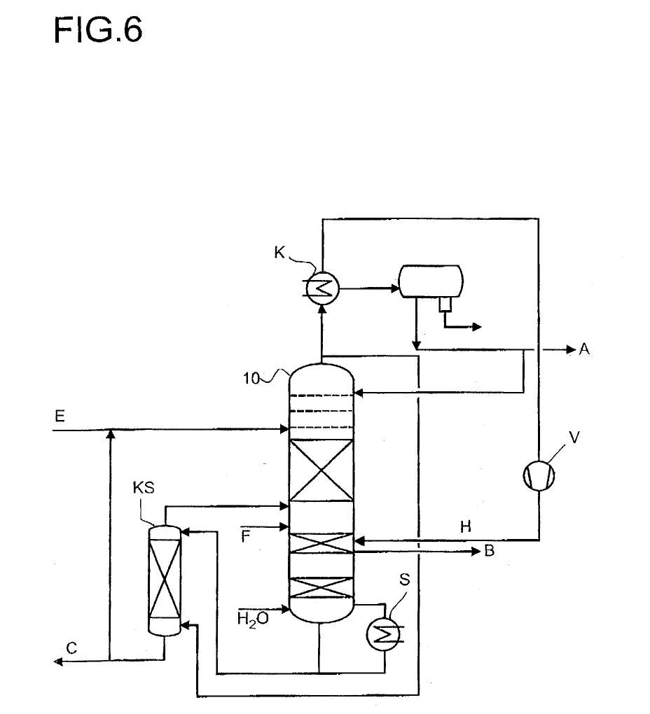

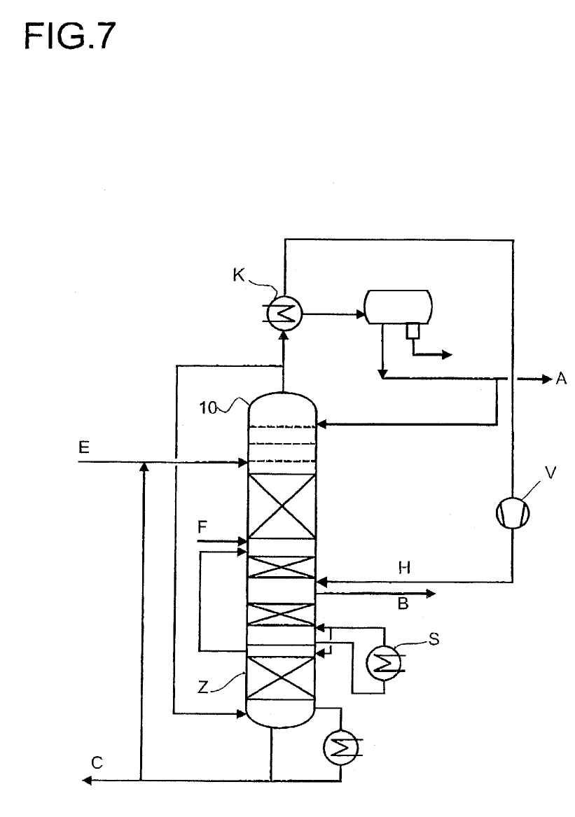

FIG. 5 schematically shows a process variant with supplying of steam, and FIGS. 6 and 7 schematically show two different process variants in which a relatively high 1,3-butadiene content is set in the column bottom.

In the figures, the same reference numerals are used for the same or corresponding streams.

The variant shown schematically in FIG. 1 has a dividing wall column TK with a dividing wall T which is arranged in the longitudinal direction of the column and divides the dividing wall column TK into an upper common column region 1, a lower common column region 6, an inflow section 2a, 2b, 4 and an offtake section 3a, 3b, 5a, 5b. The C4 fraction is introduced via feed point F between the subsections 2b and 4 of the inflow section, the extractant E is introduced between the subsections 2a and 2b of the inflow section and hydrogen H is introduced into the lower common column region 6. In the condenser K, the condensable low boilers are separated from the vapor stream, partly returned as runback to the top of the column and otherwise discharged as low boiler stream A. The gaseous hydrogen is compressed in the compressor V and fed back into the lower common column region 6 of the dividing wall column TK. The column has a bottoms vaporizer S via which part of the bottom product is returned to the lower common column region 6, and part of the bottom product is, without recirculation via the bottom vaporizer, discharged from the dividing wall column as high boiler stream C.

The inflow section of the dividing wall column TK is formed by the subsections 2a, 2b and 4, with the subsection 2a being located above the feed point for the extractant E, the subsection 2b being located between the feed points for the extractant E and the C4 fraction F, and the subsection 4 being located below the feed point for the C4 fraction F. The offtake section of the dividing wall column is formed by the subsections 3a, 3b, 5a and 5b. The subsection 5b has dimensions such that extractant from the lower common column region 6 cannot get into the subsection 5a of the offtake section which is equipped with reactive internals. The 1,3-butadiene-containing stream B is taken from the offtake section of the dividing wall column TK between the subsections 3b and 5a.

FIGS. 2a to 2d schematically show different embodiments and apparatus variants comprising thermally coupled distillation columns each having a common bottoms vaporizer and common condenser. Here, the column regions 1, 2a, 2b, 3a, 3b, 4, 5a, 5b and 6 of the dividing wall column TK of FIG. 1 are divided up differently among two individual columns.

FIGS. 3a to 3d show further embodiments of thermally coupled columns in which each column has its own bottoms vaporizer and its own condenser. The runback for each individual column is generated by condensation in its own condenser. To reduce energy consumption, the condensers are preferably designed as partial condensers.

FIG. 4 is a schematically shows a plant for carrying out the embodiment without a dividing wall, in which a middle boiler stream is supplied:

A single column 10 is supplied in its upper region with the selective solvent E, with the C4 fraction F in its middle region, and with a hydrogen stream H below the supply side of the stream F. A crude 1,3-butadiene stream B is taken off as a side stream. The column is equipped with trays in the region above the supply side of the stream E and below that point is equipped with random packing elements or ordered packings, some of which must be catalytically active. The vapor stream is condensed and taken off as a low boiler stream A, containing predominantly butanes and butenes. From the column bottom, solvent is taken off into the bottoms vaporizer S, where it is partially purified and then recycled into the stream E. A middle boiler stream C5 is supplied at the bottoms vaporizer S.

FIG. 5 schematically shows a plant for implementing the preferred process variant where a stream of water is supplied, preferably in vapor form (H2O-vap) in its lower region. This quantity of water is passed back into the column in a circuit by condensing the crude 1,3-butadiene stream and separating off the aqueous phase in a phase separator, preferably by evaporating it.

In the alternative illustrated in FIG. 6, which relates to a process where an increased 1,3-butadiene content is allowed in the bottoms liquid, the bottoms liquid is subjected to extractive stripping in a divided stripping column KS. For this purpose it is possible, as illustrated by way of example, to use the vapor stream from column 10.

FIG. 7 schematically shows a further plant for implementing a process variant with an increased concentration of 1,3-butadiene. In this variant, the stripping column KS is placed against the column 10, as an additional bottommost section Z, and is separated from said column 10 in a gastight and fluidtight manner.

### EXAMPLES

A column with a total of 70 theoretical plates, with a column-top pressure of 4.5 bar, was supplied at the 45th tray, counting from the bottom, with a volume flow of 1.5 kg/h of a C4 fraction whose composition was as indicated earlier on above. At tray 65 an aqueous solution containing NMP, with a strength of 8.3% by weight, was supplied as the selective solvent. A hydrogen stream of 15 g/h was supplied at tray 11. A crude 1,3-butadiene stream was taken off from tray 10. In the column bottom, a temperature of 186° C. became established.

Under the experimental conditions indicated above, the volume flows of the middle boiler 2-methylbutene supplied to the column bottom in each case were as set out below.

The reduction in bottoms-liquid temperature achieved in each case can be seen from the table below:

| Example | C5 volume flow | Temperature of bottoms |
|---|---|---|
| No. | (g/h) | liquid (° C.) |
| 1 | 3 | 184.6 |
| 2 | 6 | 183.4 |
| 3 | 30 | 175.2 |
| 4 | 60 | 168.1 |
| 5 | 120 | 159.2 |
| 6 | 240 | 150.2 |

### Example 7

Under the same experimental conditions as described initially, a steam stream of 300 kg/h was supplied to the lower region of the column instead of a C5 stream. This resulted in a reduction in bottoms-liquid temperature from 186 to 180° C.

### Example 8

The procedure of example 7 was repeated but with a higher volume of steam, 2010 g/h, being supplied. This lowered the temperature in the bottoms liquid to 165° C.

1. A process for the work-up of a C4 fraction, comprising the process steps
Extractive distillation (I),
Selective hydrogenation over a heterogeneous catalyst (II), a crude 1,3-butadiene stream being obtained following process steps (I) and (II) and
Distillation of the crude 1,3-butadiene stream for isolating pure 1,3-butadiene (III),
characterized in that the process steps I and II are carried out in a single column and the process step III is carried out in a second column.

2. A process for the work-up of a C4 fraction comprising the process steps
Extractive distillation (I),
Selective hydrogenation over a heterogeneous catalyst (II), a crude 1,3-butadiene stream being obtained following process steps (I) and (II) and
Distillation of the crude 1,3-butadiene stream for isolating pure 1,3-butadiene (III),
characterized in that the process steps I and II are carried out in thermally coupled columns and the process step III is carried out in a further column.

3. A process as claimed in claim 1 or 2, characterized in that the extractant used for the process step I is N-methylpyrrolidone, preferably in aqueous solution, in particular with from 8 to 10% by weight of water, particularly preferably with 8.3% by weight of water.

4. A process as claimed in any of claims 1 to 3, characterized in that the heterogeneous catalyst used for the selective hydrogenation (process step II) is a TLC packing.

5. A process as claimed in any of claims 1 to 4, characterized in that a stream is taken off from the column in which process steps (I) and (II) are conducted from a zone having a relatively high concentration of acetylenes and this stream is supplied again to the column, preferably to the topmost region of its catalytically active zone.

6. A process as claimed in any of claims 1 to 5, characterized in that at least one measure is taken to lower the temperature of the liquid in the bottom of the column.

7. A process as claimed in claim 6, characterized in that the temperature of the liquid in the bottom of the column is lowered by from 10 to 80° C., in particular to a level in the range from 100 to 170° C., preferably from 140 to 160° C.

8. A process as claimed in claim 6 or 7, characterized in that a middle boiler stream is supplied to the lower region of the column or to the bottoms vaporizer of the column.

9. A process as claimed in claim 8, characterized in that as middle boiler a substance or mixture of substances is supplied which is already present in the process.

10. A process as claimed in claim 8 or 9, characterized in that as middle boiler a substance or a mixture of substances having in each case 5 carbon atoms per molecule is supplied, preferably one or more alkanes and/or more alkenes.

11. A process as claimed in claim 10, characterized in that as middle boiler one or more of the following substances is supplied: 2-methyl-2-butene, 3-methyl-1-butene, n-pentane, isopentane, n-pent-1-ene and n-pent-2-ene.

12. A process as claimed in any of claims 8 to 11, characterized in that the ratio of the volume flow of the middle boiler to the volume flow of the C4 fraction supplied is from 0.001/1 to 0.25/1, preferably from 0.002/1 to 0.15/1, with particular preference from 0.004/1 to 0.008/1.

13. A process as claimed in either of claims 6 and 7, characterized in that the amount of the relatively low-boiling components from the selective solvent, in particular its steam content, is increased in the lower region of the column by supplying a stream of the relatively low-boiling component of the selective solvent, especially steam, to the lower region of the column and depleting the stream of selective solvent taken off from the column, prior to its partial or complete recycling to the column, by the supplied fraction of relatively low-boiling component, especially steam.

14. A process as claimed in claim 13, characterized in that the ratio of the relative volume flow of relatively low-boiling component, especially steam, to the volume flow of the C4 fraction supplied to the column is from 0.2/1 to 1.6/1, preferably 1.2:1.

15. A process as claimed in claim 13 or 14, characterized in that the relatively low-boiling component of the selective solvent, especially water, is supplied to the column in vapor form, at a pressure equal to or slightly above the bottom pressure of the column.

16. A process as claimed in claim 6 or 7, characterized in that in the bottoms liquid of the column an increased 1,3-butadiene content, in particular from 0.5 to 5% by weight based on the total weight of the bottoms liquid, preferably from 1 to 3% by weight, with particular preference 1.8% by weight is allowed and the bottoms liquid is depleted, after it has been taken off from the column, of 1,3-butadiene in a stripping column, using as stripping vapor preferably the vaporous top product of the column.

17. A process as claimed in any of claims 1, 3, 4 and 5, characterized in that the process steps I and II are conducted in a dividing wall column.

18. An apparatus for carrying out a process as claimed in claim 17 comprising
a dividing wall column (TK) in which a dividing wall (T) is arranged in the longitudinal direction of the column to form an upper common column region (1), a lower common column region (6), an inflow section (2a, 2b, 4) and an offtake section (3a, 3b, 5a, 5b),
with introduction of the C4 fraction (F) in the middle region of the inflow section (2a, 2b, 4), between the subsections (2b and 4) of the latter, introduction of extractant (E) in the upper region of the inflow section (2a, 2b, 4) between the subsections (2a and 2b),
with introduction of hydrogen (H) below the subsection (5a), separation of unreacted hydrogen in the vapor stream of the dividing wall column (TK) from condensable low boilers in a condenser (K) and recirculation via a compressor (V) to the lower common column region (6) and
with discharge of the 1,3-butadiene-containing stream (B) from the offtake section (3a, 3b, 5a, 5b) of the dividing wall column (TK) at a point between the subsections (3b) and (5a)
further transport of the stream (B) to the final distillation (process step III).

19. An apparatus as claimed in claim 18, characterized in that the low boilers condensed from the vapor stream in the condenser (K) are partly returned as runback to the top of the dividing wall column (TK) and otherwise discharged as low boiler stream (A).

20. An apparatus as claimed in claim 18 or 19, characterized in that the dividing wall column (TK) has reactive internals in the subsection (5a) of the offtake section (3a, 3b, 5a, 5b) located between the point corresponding to the feed point (F) and the point of discharge of the 1,3-butadiene-containing stream (B) from the offtake section of the dividing wall column (TK).

21. An apparatus as claimed in claim 20, characterized in that reactive internals are present in addition in the upper subsections of the offtake section (3a, 3b, 5a, 5b), preferably in the subsection (3b), particularly preferably in the subsections (3a and 3b).
